(19) United States
(12) Patent Application Publication
(10) Pub. No.: US 2009/0093668 A1
(43) Pub. Date: Apr. 9, 2009

(54) Device to stent an airway during brachytherapy treatment and to provide a secure and precise positioning of a treatment radiotherapy capsule within airway

(75) Inventors: Lewis H. Marten, Westwood, MA (US); Dennis F. Creedon, Sandwich, MA (US)

Correspondence Address: FROMMER LAWRENCE & HAUG, 745 FIFTH AVENUE- 10TH FL., NEW YORK, NY, 10151, US

(73) Assignee: E. Benson Hood Laboratories

(21) Appl. No.: 11/906,917

(22) Filed: Oct. 4, 2007

(51) Int. Cl. A61M 36/12

(52) U.S. Cl. 600/7

(57) ABSTRACT

This invention relates to a device for brachytherapy treatment of a tissue growth on the inner surface of an airway lumen. The invention also relates to a device for treatment of an obstructing tissue growth on the inner surface of an airway lumen. The device is comprised of a cylindrical tube having a proximal end, a distal end, and a continuous open cavity therebetween, wherein the device is deployed against the inner surface of the lumen. There is at least one longitudinal channel contained within the wall of the tube for positioning a treatment radiotherapy source in close proximity to said tissue growth. The present invention also discloses a method for treating a tissue growth on the inner surface of an airway lumen, and method for treating an obstructing tissue growth on the inner surface of an airway lumen

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to a device for brachytherapy treatment of a tissue growth on the inner surface of an airway lumen. The invention also relates to a device for treatment of an obstructing tissue growth on the inner surface of an airway lumen. The device is comprised of a cylindrical tube having a proximal end, a distal end, and a continuous open cavity therebetween, wherein the device is deployed against the inner surface of the lumen. There is at least one longitudinal channel contained within the wall of the tube for positioning a treatment radiotherapy source in close proximity to said tissue growth.

In addition, the present invention also discloses a method for treating a tissue growth on the inner surface of an airway lumen, and method for treating an obstructing tissue growth on the inner surface of an airway lumen

### BACKGROUND OF THE INVENTION

Abnormal tissue growth, or tumors, can form within the lumen wall of an airway. The growth is typically due to the formation of a primary tumor, the invasion of an adjacent tumor, or the development of metastatic disease. Primary airway tumors, which originate within the lumen wall of the airway, are primarily either squamous cell carcinoma, which begins in flat cells lining the airway, or anadnoid cystic carcinoma, which begins in cells that make and release mucus and other fluids within the lumen. Adjacent tumors occur outside of the airway, but invade through the wall of the airway and produce endoluminal disease. The adjacent tumors are most commonly associated with lung cancer, but can also stem from esophageal, thyroid, or mediastinal malignancies. Metastatic disease refers to cancer that originates in other sites but spreads to the airway and produces an endoluminal tumor. Such cancers include renal cell carcinoma, sarcoma, breast cancer, or colon cancer.

One of the most dangerous risks associated with tissue growth within the airway is the development of malignant airway obstruction. This can lead to dyspnea, stridor, and obstructive pneumonia, and potentially even suffocation. Obstruction of the distal airway, namely the mainstem bronchi or bronchus intermedius, can also lead to progressive loss of unilateral lung function.

Treatments for malignant airway obstruction are often directed to immediate palliation by suppression or removal of the abnormal tissue growth. Such treatments include airway dilatation, mechanical core-out, laser ablation, cryotherapy, photodynamic therapy, and insertion of a stent. Airway dilatation involves passing a device, such as a rigid bronchoscope, beyond the growth, which causes compression of the growth against the lumen wall. However, with this technique, the increased patency of the lumen is only temporary, and may last only a few hours.

Mechanical core-out and laser ablation are methods that cut away part of the tissue growth to re-establish the patency of the lumen. Mechanical core-out involves shearing off large pieces of the tissue growth, while laser ablation essentially burns off tissue. However, both techniques are associated with the risk of perforating the lumen wall, and laser ablation can lead to an airway fire or skin burns. Further, neither technique prevents endoluminal tissue regrowth.

Other techniques include cryotherapy and photodynamic therapy, which both involve necrotizing the abnormal tissue growth. Cryotherapy is based on freezing the tissue, while photodynamic therapy involves the administration of a photosensitizing agent to the tissue, followed by activation of the agent with light of a specific wave length. Unfortunately, both techniques do not result in immediate removal of the tissue growth, as the tissue requires time to necrotize. Cryotherapy additionally requires complex equipment and can be very time consuming to apply to the tissue growth. On the other hand, photodynamic therapy may cause an initial worsening of the obstruction due to edema, mucous plugs, or atelectasis, and the patient may have to avoid sunlight since the photosensitizing agent can last for four to six weeks.

Stents are devices that are placed at the obstruction site to physically force the tissue growth against the lumen wall, thereby providing immediate palliation. Notably, stents do not treat the tissue growth itself; if the growth is a tumor, stents will not prevent the tumor from spreading throughout the lumen wall and the lumen itself.

Alternatively, brachytherapy can be used to effectively treat abnormal tissue growths, notably tumors. Brachytherapy is defined as internal radiation therapy given by placing a radioactive source directly into or near a tumor. It is performed in the airway by bronchoscopically placing a hollow catheter into the trachea or bronchus directly adjacent to the malignant lesion. A radioactive source is then advanced on a guide wire into the catheter and positioned next to the lesion for a period of time to treat the malignant lesion. The intent is to get the capsule, which contains the radioactive material, positioned as close as possible to the tumor site. The active positioning of the radioactive capsule is crucial in order to deliver a precise and controlled radioactive exposure to the cancerous tissue and minimal exposure to the healthy tissue. Brachytherapy is generally less invasive than surgery, usually results in fewer side effects than surgery or external beam radiation, allows for a shorter recovery time, and reduces the impact on the patient's quality of life.

However, brachytherapy does not provide a means of immediately palliating the obstructed airway. Further, current brachytherapy practices often involve leaving a thin floppy catheter loosely positioned in a large caliber tracheal lumen adjacent to a small lesion on the wall. This attempt to treat the cancerous tissue, with a relatively uncontrolled exposure to the target tissue, is coupled with an opportunity to expose healthy tissue to unnecessary radiation.

There are few patented devices used in brachytherapy treatments directed to the positioning of the radioactive source within a passageway. U.S. Pat. No. 6,994,688 relates to a catheter which has an attachment that can house a radiotherapy source to be introduced into the body. However, the device does not precisely control whether healthy tissue is exposed to the radiotherapy source. U.S. Pat. No. 6,607,476 relates to an apparatus for positioning a radiotherapy source in a passageway, wherein the apparatus includes a reconfigurable positioning element through which blood may flow when in an expanded state. While the apparatus does not inhibit movement in the passageway, there is not a precise means to position the radiotherapy source next to the tissue growth. U.S. Pat. No. 6,746,392 relates to a transluminal, over-the-wire catheter that provides a lumen for guiding a radiation source wire to an intended treatment site within a patient. The catheter of the invention includes a first lumen for the guidewire and a second lumen for the radiation source wire, and the two lumens are twisted together to form the desired parallel double helix configuration for the guidewire and the radiation source wire. The incorporation of the radiation source into the wire, however, prevents accurate positioning of said source. U.S. Pat. No. 6,379,380 relates to a metal stent comprising a generally tubular structure wherein the metal contains a substantially uniform dispersion of enriched stable isotopes; when activated, the stent emits low to moderate dosages of radiation. Notably, this device will irradiate healthy as well as the target tissue. Finally, U.S. Pat. No. 5,916,143 relates to a catheter apparatus which contains a plurality of discrete balloon sections. When inflated with radioactive gas, the balloons conform to the targeted tissue and provide homogenous radiation delivery. Importantly, the tissues irradiated by the apparatus cannot be precisely controlled.

Clearly, there is not an effective treatment of tissue growths in airway lumens which can precisely position a radiotherapy source next to growth. Further, there is not a means to both effectively treat and provide immediate palliation of a tissue growth obstructing an airway lumen.

Citation or identification of any document in this application is not an admission that such document is available as prior art to the present invention.

### SUMMARY OF THE INVENTION

The present invention is directed to a device for brachytherapy treatment of a tissue growth on the inner surface of an airway lumen, wherein the device can precisely position a radiotherapy source in close proximity to the tissue growth.

Another aspect of the invention is directed to a device for brachytherapy treatment of a tissue growth obstruction on the inner surface of an airway lumen, wherein the device can both provide pressure against the growth to re-establish patency of the lumen, and precisely position a radiotherapy source in close proximity to the tissue growth.

Yet another aspect of the invention is a method for treating a tissue growth on the inner surface of an airway lumen by deploying the brachytherapy treatment device within said lumen and precisely positioning a radiotherapy source in close proximity to the tissue growth.

Further, another aspect of the invention is a method for treating a tissue growth obstruction on the inner surface of an airway lumen, wherein the method comprises deploying the brachytherapy treatment device within said lumen at the site of the obstruction and precisely positioning a radiotherapy source in close proximity to the tissue growth.

The device of the present invention is comprised of a cylindrical tube having a proximal end, a distal end, and a continuous open cavity therebetween, wherein the tube is deployed against the inner surface of the airway lumen. There is at least one longitudinal channel contained within the wall of the tube for positioning a treatment radiotherapy source in close proximity to said tissue growth.

In one embodiment, the cylindrical tube can maintain the patency of the lumen by applying pressure against the tissue growth.

In a preferred embodiment, there are multiple channels within the wall of the tube. In another preferred embodiment, the channel is coated with a protective polymer. In yet another preferred embodiment, the proximal end of the channel is chamfered.

In one embodiment, the device can be used to treat multiple tissue growths on the inner surface of the airway lumen. In another embodiment, the device can be used to treat multiple tissue growths on the inner surface of the airway lumen, wherein the tissue growth is obstructing the lumen.

The outer surface of the cylindrical tube can contain a plurality of protrusions as a means for anchoring the tube to the airway. In a preferred embodiment, the protrusions are distributed along the length of the cylindrical tube. In another preferred embodiment, the protrusions are positioned at 90° to each other within a common cross-sectional plane.

The device can be molded out of a polymer, preferably silicone rubber.

Furthermore, the inner and outer surfaces of the cylindrical tube are preferably coated with a protective polymer.

In one embodiment, the radiotherapy source is provided in the form of microspheres, capsules, pellets, fiber, ribbon, mesh, patch and film. In a preferred embodiment, the radiotherapy source is a photon or gamma source. In yet another preferred embodiment, the radiotherapy source is selected from the group consisting of Iridium-192, Palladium-103, and Iodine 125.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawing, in which:

FIG. 1 shows a cross-sectional view and a side view, including the longitudinal channels, of a preferred embodiment of the invention.

FIG. 2 shows a magnified cross-sectional view and a magnified side view, including the longitudinal channels, of a preferred embodiment of the invention.

### DETAILED DESCRIPTION

The present invention is a device for brachytherapy treatment of a tissue growth on the inner surface of an airway lumen. The device is comprised of a cylindrical tube that is deployed against the inner lumen wall at the site of the growth. The walls of the cylindrical tube contain at least one longitudinal channel for guiding, positioning, and anchoring the radiotherapy source. The channel provides a means to position the radiotherapy source in close proximity to the tissue growth in order to irradiate the growth.

The present invention can also be used for brachytherapy treatment of a lumen having multiple tissue growths. The walls of the cylindrical tube can contain multiple longitudinal channels, wherein more than one radiotherapy source can be guided into the channels that are in closest proximity to the tissue growths, or more than one radiotherapy source can be guided into the same channel, or some combination thereof.

The present invention is also a device for brachytherapy treatment of a tissue growth on the inner surface of an airway lumen which is obstructed by the growth. The device is comprised of a cylindrical tube that is deployed against the inner surface of the lumen at the site of the obstruction. The tube applies pressure against the tissue growth, thereby preventing the growth from obstructing the lumen. The walls of the cylindrical tube contain at least one longitudinal channel for guiding, positioning, and anchoring the radiotherapy source. The channel provides a means to position the radiotherapy source in close proximity to the tissue growth in order to irradiate the growth's cells.

Further, the present invention can also be used for brachytherapy treatment of more than one tissue growth on the inner surface of an airway lumen which is obstructed by the growths. The cylindrical tube, when deployed in the lumen, can apply pressure against the tissue growths. The walls of the cylindrical tube can contain multiple longitudinal channels, wherein more than one radiotherapy source can be guided into the channels that are in closest proximity to the tissue growths, or more than one radiotherapy source can be guided into the same channel, or some combination thereof.

The tissue growth can be a tumor or multiple tumors. For example, the growth may be a primary airway tumor which forms within the airway. Alternatively, the growth may be due to an adjacent tumor that invades through the wall of the airway lumen, thereby producing obstructing endoluminal disease. The growth may be also stem from metastatic disease, wherein the growth is due to remote primary tumors such as, but not limiting to, renal cell carcinoma, sarcoma, breast cancer, or colon cancer.

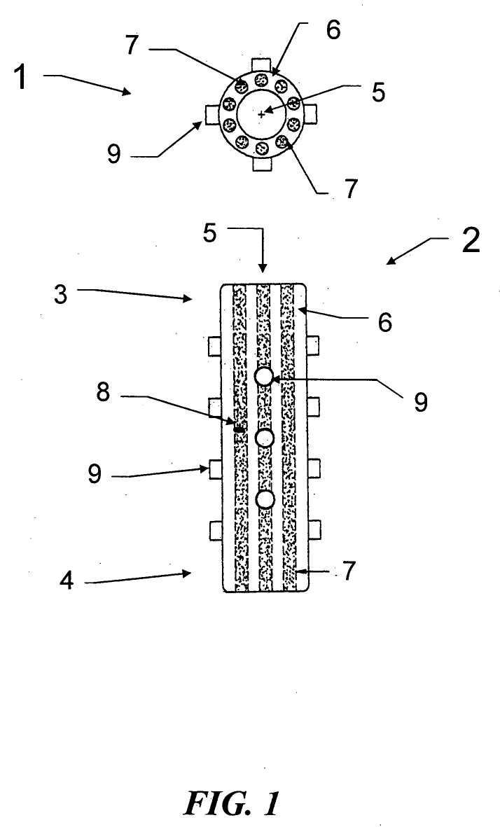

FIG. 1 illustrates a preferred embodiment of the present invention in a cross-sectional view 1 and a side view 2, wherein the longitudinal channels are shown. The invention is comprised of a cylindrical tube, which has a proximal end 3, distal end 4, and a continuous open cavity therebetween 5. Within the wall 6 of the tube, there are multiple channels 7 that run longitudinally which are used for guiding, positioning, and anchoring the radiotherapy source 8. There is also a plurality of protrusions 9 extending radially from the outer surface of the tube. The protrusions 8 are distributed around the outer circumference and along the length of the tube.

The outer diameter of the tube is sized according to the inner circumference of the airway lumen wherein the tube will be deployed. The outer diameter of the tube can range from 10 mm to 20 mm, preferably 14 mm to 18 mm. The thickness of the cylinder wall can vary but must accommodate the longitudinal channel(s), as well as allow ample passage of air/fluid through the inner cavity of the tube. The wall thickness can range from 2 mm to 4 mm, preferably 2.75 mm to 3.25 mm, most preferably 3 mm. Thus, the inner diameter can range from 4 mm to 16 mm, but preferably, 8 mm to 12 mm.

The length of the cylindrical tube is variable, but must appropriately encompass the site(s) of the tissue growth(s). Thus, the length can range from 20 mm to 60 mm, preferably 35 mm to 45 mm.

In one embodiment, the dimensions of the cylindrical tube can be uniform throughout its length. In another embodiment, the dimensions can vary, based on the site of the tissue growth and the size and shape of the airway lumen at that site.

The channels in the wall of the tube can vary in number, distribution, and in size. The number of channels is at least one, but the maximum value is limited by the circumference of the cylindrical tube. Preferably, the tube wall will contain 4-16 longitudinal channels. The channels are preferably spaced evenly around the circumference of the tube. Further, the size of the channels can vary, although the preferred diameter is 1 mm to 2.5 mm, more preferably 1.5 mm to 2 mm, most preferably 1.85 mm.

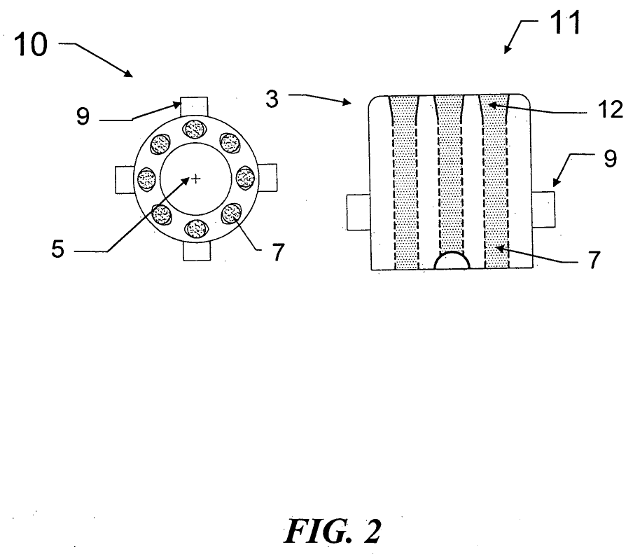

FIG. 2 is a preferred embodiment of the invention illustrating a magnified cross-sectional view 10 and a magnified side view 11, wherein the longitudinal channels are shown. The proximal end of the longitudinal channel(s) 12 are preferably chamfered or bedeviled, which provides a means of guiding the leading edge of the catheter assembly into the appropriate channel for positioning of the radiotherapy source.

The protrusions radiating from the tube can anchor the device within the airway lumen. The protrusions can vary in size, and may extend from 0.5 mm to 4 mm, preferably 1.5 mm to 2.5 mm, most preferably 2 mm, from the surface of the cylindrical tube. The protrusion can also vary in shape, but is preferably circular. The width/diameter of the protrusions can range from 0.5 mm to 6 mm, preferably 2.5 mm to 3.5 mm, most preferably 3 mm.

In one embodiment, the device can have protrusions of the same size and shape. In another embodiment, the invention can have protrusions of varying size and shape, based on the site of the tissue growth and the size and shape of the airway lumen at that site.

The protrusions are oriented around the circumference of the tube, although these orientations can vary. Within a common cross-sectional plane, the protrusions can be positioned at 30°, 45°, 60°, 900°, and 120°, preferably 90°. The protrusions are also arranged along the length of the tube, but the arrangement can vary. For example, the protrusions can be in a straight line, in an angled or curved line, or in a spiral-like arrangement.

The device is molded out of a material, preferably a polymer, that offers soft and pliable properties. These properties allow for ease of insertion of the device into the airway lumen by means of folding the device into an introducer tube. The material also allows for compression of the device during removal after the therapy regime is complete. Materials can include, but are not limited to, nitrile rubber, synthetic latex (which has no proteins), polyvinyl chloride (PVC), styrenic elastomers, polyurethane, polyethylene terephthalate (PET), high density polyethylene (HDPE), polycarbonate urethane (PCU), polytetrafluoroethylene (PTFE), expanded polytetrafluoroethylene (ePTFE), polyolefins, polypropylene, and silicone. The preferred material is silicone.

The inner surface of the multiple channels has a protective polymeric coating. This coating facilitates the introduction and passage of the catheter into the lumen. Further, the coating prevents accumulation of mucus and granulation tissue between treatment sessions. The protective coating is also applied to the exterior and interior wall of the device in order to prevent the accumulation of mucus, crusting, and granulation on or around the device. The protective polymer can be a polymer including, but not limited to, parylene, polyurethane, polyvinyl pyrrolidine, polyvinyl alcohol, phosphorylcholine, silanes, urethane, polycaprolactone (PCL), polymethylmethacrylate (PMMA), combinations of the above, and the like.

The radiotherapy source may be any suitable radioactive material known for use in therapeutic treatment of the human or animal body. Preferred radioactive sources are gamma and/or beta-emitting sources. Examples of suitable radioactive sources include, but are not limited to, Iodine-125, palladium-103, strontium-90, ruthenium-106, phosphorus-32, samarium-145, iridium-192, cobalt-60, radioactive vanadium-48 and yttrium-90. The radioactive source may be selected based on the specific needs of the particular treatment process, the half-life, the amount of radiation required and other parameters, as known in the art.

In another embodiment, the radiation source material further comprises a diluent. The diluent can be added during, prior to, or after the purification process. Suitable diluents for radiotherapy sources may include platinum metal, palladium metal, rhodium metal, or any other suitable material which is compatible with the radiation released by the radiotherapy source. More diluents are biocompatible materials or certain polymeric materials which can be employed by, for example, homogeneously mixing the radiation source material with the polymer prior to its application to the substrate, or even by carrying out such mixing and using the mixture of polymeric material and radiation source material as the substrate itself. The amount of diluent added would be well known to one skilled in the art.

Another aspect of the invention is a method of treating a tissue growth or multiple tissue growths on the inner surface of an airway lumen; alternatively, the invention can be a method for treating a tissue growth or multiple tissue growths on the inner surface of an airway lumen, wherein the lumen is obstructed by the growths.

In both methods, the device is initially folded within an introducer tube covering. The device is then inserted into the lumen to the site of the tissue growth or tissue growths. The insertion preferably occurs using a ridged bronchoscope. The method of using a ridged bronchoscope is well known in the art.

The device is deployed in the airway lumen by removing the introducer tube covering. The device forms a cylindrical tube shape within the lumen, such that the outer surface of the tube is pressed up against the inner surface of the lumen. The device is applying pressure to the inner surface of the lumen, which allows it to maintain its position within the lumen. Further, if tissue is obstructing the lumen, the pressure will push the obstructing tissue back against the inner lumen wall. In a preferred embodiment, the device contains protrusions which contact the inner lumen surface to anchor the device in place and allow for natural movement/flow of mucus and air over the airway wall. In an even more preferred embodiment, the resulting effect of anchoring the device to the airway inner lumen surface would be a positive healing of the treated tissue.

A catheter is used to guide the radiotherapy source into the appropriate channel and is used to position the radiotherapy source at the appropriate distance into the channel such that it is as close in proximity to the tissue growth as possible. If there are multiple tissue growths, then radiotherapy sources are placed into multiple channels or more than one source is placed in the same channel, or a combination thereof, depending on the locations of the multiple tissue growths. Once the tissue growth has been exposed to the radiotherapy source for the appropriate amount of time, as determined by what is known in the field, and the treatment session is complete, the radiotherapy source is removed.

Between treatment sessions, the device can stay in the airway lumen, as the open cavity within the cylindrical tube allows for the free passage of air/fluids. Once the therapy regime is complete, the device is compressed and removed from the lumen, preferably by refolding the device into the introducer tube covering.

Having thus described in detail preferred embodiments of the present invention, it is to be understood that the invention is not to be limited to particular details set forth in the above description as many apparent variations thereof are possible without departing from the spirit or scope of the present invention.

What is claimed is:

1. A device for brachytherapy treatment of a tissue growth on the inner surface of an airway lumen, wherein the device comprises:
(a) a cylindrical tube having a proximal end, a distal end, and a continuous cavity; therebetween, wherein the tube is deployed against the inner circumference of the lumen; and
(b) at least one longitudinal channel contained within the wall of the tube for positioning a treatment radiotherapy source in close proximity to said tissue growth.

2. The device according to claim 1, wherein the cylindrical tube maintains the patency of the lumen by applying pressure against the tissue growth.

3. The device according to claim 1, wherein the tissue growth is a tumor.

4. The device according to claim 4, wherein the tissue growth is comprised of multiple tumors.

5. The device according to claim 1, wherein the walls of the tube contain multiple channels around the circumference of the tube.

6. The device according to claim 1, where the channels are coated with a protective polymer.

7. The device according to claim 1, wherein the channels are chamfered on the proximal end.

8. The device according to claim 1, wherein the outer surface of the cylindrical tube contains a plurality of protrusions as a means for anchoring the tube to the airway.

9. The device according to claim 8, wherein the protrusions are distributed along the length of the cylindrical tube.

10. The device according to claim 8, wherein the protrusions are positioned at 90° to each other within a common cross-sectional plane.

11. The device according to claim 1, wherein the cylindrical tube is made out of polymer.

12. The device according to claim 11, wherein the polymer is silicone rubber.

13. The device according to claim 1, wherein the inner and outer surfaces of the cylindrical tube are coated with a protective polymer.

14. The device according to claim 1, wherein the treatment radiotherapy source is provided in a form selected from the group consisting of microspheres, capsules, pellets, fiber, ribbon, mesh, patch and film.

15. The device according to claim 14, wherein the radiotherapy source is a photon or gamma source.

16. The device according to claim 15, wherein the radiotherapy source is selected from the group consisting of Iridium-192, Palladium-103, and Iodine 125.

17. A device for brachytherapy treatment of an obstructing tissue growth on the inner surface of an airway lumen, wherein the device comprises:
(a) a cylindrical tube having a proximal end, a distal end, and a continuous cavity therebetween, such that, when deployed, it applies pressure against the inner circumference of the lumen and the tissue growth; and
(b) at least one longitudinal channel contained within the wall of the tube for positioning a treatment radiotherapy source in close proximity to said tissue growth.

18. A method for treating a tissue growth on the inner surface of an airway lumen, comprising:
(i) deploying the device of claim 1 into the lumen at the site of the tissue growth; and
(ii) positioning the radiotherapy source within the appropriate channel such that the radiotherapy source is adjacent to the tissue growth.

19. The method of claim 18, wherein the tissue growth is obstructing the airway lumen.
